(19) United States
(12) Patent Application Publication
(10) Pub. No.: US 2015/0104508 A1
(43) Pub. Date: Apr. 16, 2015

(54) DEVICES, COMPOSITIONS, AND METHODS FOR FABRICATING DRUG DELIVERY SYSTEMS

(71) Applicant: Zzyzx Polymers LLC, Allentown, PA (US)

(72) Inventors: Philip BRUNNER, East Stroudsburg, PA (US); Mark TAPSAK, Orangeville, PA (US)

(21) Appl. No.: 14/511,688

(22) Filed: Oct. 10, 2014

Related U.S. Application Data
(60) Provisional application No. 61/890,185, filed on Oct. 12, 2013.

(51) Int. Cl. A61J 3/02; A61K 9/14

(52) U.S. Cl. CPC A61J 3/02; A61K 9/146
USPC 424/464; 424/489; 424/501; 424/499; 241/3; 241/66; 241/67; 264/238

(57) ABSTRACT

Systems, methods, compositions, and devices related to the delivery of one or more biologically active agents to a body include the admixture of one or more biologically active agents with one or more biocompatible polymeric mixtures in a solid-state shear extrusion system. The extrusion systems may include one or more extrusion screws. One or more portions of the one or more extrusion screws, one or more extrusion system active elements, one or more barrel sections, and/or one or more extruder work zones may be temperature controlled to maintain a temperature of the biocompatible polymeric mixture in contact therewith at or below the liquefication temperature of the biocompatible polymeric materials. The resulting compositions from the extrusion systems may be fabricated into devices to deliver the one or more biologically active agents to a body.

### CLAIM OF PRIORITY

This application claims benefit of and priority to U.S. Provisional Application No. 61/890,185 entitled “Method to Prepare Drug Delivery Systems using Solid-State Shear Pulverization or Solid-State Melt Extrusion” filed Oct. 12, 2013, the disclosure of which is incorporated by reference herein in its entirety.

### BACKGROUND

Delivery systems of biologically active agents are designed to administer a therapeutically effective amount of one or more biologically active agents to an organ or tissue of a subject at a controlled delivery rate. In some instances, these systems may deliver the compounds over an extended period of time such as over hours, days, or months. As one example of a desirable long-term delivery of a biologically active agent, a biologically active agent delivery composition may be incorporated in a wound dressing for control of a biologically active compound to and release of compounds from the wound site during the course of wound healing. For example, biologically active agents may be delivered over time to a wound in order to control the growth of pathogens. Such biologically active agent delivery systems may include one or more biologically active agent delivery compositions. Biologically active agent delivery compositions may include one or more biologically active agents mixed with or imbedded within one or more matrix materials.

Twin-screw extrusion (TSE) has long been established as a technique for mixing polymer blends, polymer composites, and/or polymer nanocomposites. However, TSE may be unsuitable for combining biologically active agents with polymer matrix materials to form biologically active agent delivery compositions. The shear mixing in TSE is often performed under temperature conditions sufficiently high to maintain the polymer components in the melted state. Such high temperatures may degrade biologically active agents. Furthermore, the long period of exposure to high temperatures resulting from local frictional heating of the agents can accelerate this degradation. Additionally, fillers, binding agents, excipients, and other additives that may be used to control the delivery rate of the agents may not be dispersed effectively throughout the polymer matrix using TSE processes. These limitations may render TSE ineffective for producing biologically active agent delivery systems fabricated from such matrix materials, fillers, and agents. Therefore, a method of compounding one or more biologically active agents with one or more matrix materials, fillers, binding agents, excipients, and other additives may require techniques other than those provided by TSE methods.

### SUMMARY

As used herein, the term “liquefication” refers to a phase transition of a polymer material from a solid state to a softened, liquid, or near-liquid state. A “liquefication temperature” refers to a temperature at which the polymer material transitions from a solid state to a softened, liquid, or near-liquid state. For a semi-crystalline polymer, a “liquefication temperature” may correspond to a melting point temperature. For an amorphous polymer, a “liquefication temperature” may correspond to a glass transition temperature. Some polymers may exist as combinations or admixtures of semi-crystalline and amorphous phases, and therefore the “liquefication temperature” may refer to either a melting point temperature or a glass transition temperature depending on the material composition.

As used herein, “administration of a biologically active agent to a subject” refers to any route of introducing or delivering the agent to a subject so that it may perform its intended function. Administration can be carried out through any suitable route, including orally, intranasally, parenterally, intravenously, intramuscularly, intraperitoneally, subcutaneously, or topically. Administration may include self-administration or the administration of the agent by another.

As used herein, the term “delivery system” refers to an article in any form, shape, or combination thereof configured to deliver one or more biologically active agents to a subject. Non-limiting examples of shapes may include films, tubing, foams, or any monolithic shape constructed of the compositions disclosed herein. As one illustrative example, a delivery system may include an injection molded component of a device, such as a connector head of a pacemaker or extruded polyurethane tubing as part of a pacemaker lead. Another non-limiting example may include a coating applied to a stent.

As used herein, the term “biostable” refers to the property of being resistant to degradation by processes that may be encountered in vivo. Thus, a biostable material may be a polymer that is resistant to degradation in vivo, such as a polymer resistant to homolytic cleavage of the polymer backbone. Some non-limiting examples of biostable materials may include medical grade silicone rubber, polyurethane, polyolefins such as polyethylene and polypropylene, polyamides, polyether ether ketone, and polyesters. Biostable materials are typically stable over the lifetime of the use of the device. Non-limiting examples of device lifetimes may include about 1 year for a glucose sensor and about 20 years for cardiac pacemaker leads.

As used herein, an “implantable medical device” refers to any type of appliance that is totally or partly introduced into a subject's body or by medical intervention into a natural orifice, and which is intended to remain in situ after the procedure. In some non-limiting examples, the duration of implantation may be essentially for the remaining lifespan of the subject. In other non-limiting examples, the duration of implantation may be temporary. For such temporary implantable medical devices, the lifetime of the implantable device may be limited by the anticipated degradation of the device in situ or its physical removal. Examples of implantable medical devices may include, without limitation, implantable cardiac pacemakers and defibrillators, leads and electrodes for such pacemakers/defibrillators, implantable organ stimulators (including but not limited to nerve, bladder, sphincter, and diaphragm stimulators), cochlear implants, prostheses, vascular grafts, self-expandable stents, balloon-expandable stents, stent-grafts, grafts, artificial heart valves, and cerebrospinal fluid shunts. In some embodiments, implantable medical devices may be administered in one or more of a vascular space, a peritoneal space, a portion of striated muscle, a portion of mucosal tissue, and an optical tissue. Additionally, implantable medical devices may be administered in a natural bodily cavity including intrauterine and rectal administration. In some embodiments, implantable medical devices may include breast and penile implants, cosmetic or reconstructive implants, devices for cell transplantation, drug delivery devices, and electrical signaling or delivery devices. It may be understood that an implantable medical device designed for the localized or systemic delivery of a biologically active agent may be within the scope of such implantable medical devices.

As used herein, the term “therapeutically effective amount” refers to a quantity sufficient to achieve a desired therapeutic and/or prophylactic effect. One non-limiting example of a therapeutically effective amount may include an amount that may result in the prevention of, or a decrease in, symptoms associated with an inflammation due to wound healing. A therapeutically effective amount of a composition administered to a subject may depend on the type and severity of the disease and on the characteristics of the individual, such as general health, age, sex, body weight, genetic predisposition, and tolerance to drugs. The amount may also depend on the degree, severity, and type of disease. One having ordinary skill in the art will be able to determine appropriate dosages depending on these and other factors. The compositions may also be administered in combination with one or more additional therapeutic compounds. As one non-limiting example, a substitute vitreous material may be administered intravitreally in addition to a pharmaceutical material to a subject having one or more signs or symptoms of an ophthalmic condition. In another example, a “therapeutically effective amount” of an anti-inflammatory drug may be an amount at which the response to an inflammatory event or source of inflammation (e.g., an implanted medical device) may be at least ameliorated.

The term “subject” refers to any animal that can benefit from the administration of the disclosed devices. Thus, subjects may include, without limitation, one or more mammals such as a human, a primate, a dog, a cat, a horse, a cow, a pig, and a rodent. In some embodiments, the subject may be a human. The subjects may be normal, healthy subjects or subjects having, or at risk for developing, a particular biological disease or condition. By way of example only, the subject may be a subject having, or at risk for developing, a foreign body reaction upon implantation of a medical device.

As used herein, the term “biologically active agent” refers to a material that exhibits biological activity in an animal. In some embodiments, the biologically active agent may be composed of small molecules having a molecular weight of less than about 1500 g/mole. Among other non-limiting embodiments, a biologically active agent may include drugs, pro-drugs, vitamins, and cofactors. In still other non-limiting embodiments, a biologically active agent may include macro-molecules including, but not limited to, proteins, nucleic acids, macrolides, and other polymers.

As used herein, a “matrix material” refers to a biocompatible material that may be mixed or compounded with a biologically active agent to form a composition that may be used at least in part to form an implantable medical device. The matrix material may include any biologically compatible material including polymers. Such matrix materials may be used to control the delivery of the biologically active agent to the subject or may form a mechanical reservoir to locate the implantable medical device at a specific tissue site within the subject.

In an embodiment, a method of fabricating a biologically active agent delivery composition may include introducing a polymeric mixture into an extruder, introducing a biologically active agent into the extruder, solid-state shearing the polymeric mixture and the biologically active agent together in an initial zone of the extruder to yield the biologically active agent delivery composition, in which the initial zone has a temperature less than or equal to a liquefication temperature of the polymeric mixture, and dispensing the biologically active agent delivery composition in a particulate form from the extruder.

In an embodiment, a biologically active agent delivery composition may include a polymeric mixture and a biologically active agent, in which the biologically active agent delivery composition is a granular material having an average particle diameter less than or equal to about 100 μm, and is fabricated by a solid-state shearing device operating at least in part at a temperature less than or equal to a liquefication temperature of the polymeric mixture.

In an embodiment, a biologically active agent delivery device may include a biologically active agent delivery composition composed of a polymeric mixture and a biologically active agent, in which the biologically active agent delivery composition is a granular material having an average particle diameter less than or equal to about 100 μm, and is fabricated by a solid-state shearing device operating at least in part at a temperature less than or equal to a liquefication temperature of the polymeric mixture, and in which the biologically active agent delivery composition is fabricated into the biologically active agent delivery device configured for administration into a portion of a body.

In an embodiment, a method of fabricating a biologically active agent delivery device may include introducing a polymeric mixture into an extruder, introducing a biologically active agent into the extruder, solid-state shearing the polymeric mixture and the biologically active agent together in an initial zone of the extruder to yield the biologically active agent delivery composition, in which the initial zone has a temperature less than or equal to a liquefication temperature of the polymeric mixture, dispensing the biologically active agent delivery composition from the extruder, and fabricating the biologically active agent delivery device from the biologically active agent delivery composition.

In an embodiment, a system for fabricating a biologically active agent delivery composition may include at least one barrel section, at least one extrusion screw disposed within the at least one barrel section, a plurality of active elements disposed within the at least one barrel section, wherein the active elements are configured to be operated by the at least one extrusion screw, at least one feed chute configured to deliver one or more of a polymeric mixture and a biologically active agent into the at least one barrel section, and a temperature control system, in which the temperature control system is configured to maintain a temperature of one of more of the one or more barrel sections, the one or more extrusion screws, and the one or more active elements less than or equal to a liquefication temperature of the polymeric mixture.

### BRIEF DESCRIPTION OF THE DRAWINGS

FIG. 1 illustrates a solid state shear pulverizer (SSSP) screw assembly in accordance with some embodiments.

FIG. 2 illustrates a solid state melt extruder (SSME) screw assembly in accordance with some embodiments.

FIG. 3 is a flow chart of an embodiment of a method of fabricating a biologically active agent delivery composition.

FIG. 4 is a flow chart of an embodiment of a method of fabricating a biologically active agent delivery device.

### DETAILED DESCRIPTION

As disclosed above, twin-screw extrusion (hereafter, “TSE”) techniques may be useful for processing homo-polymers, copolymers, and polymer blends. However, the conditions under which TSE processing may occur can limit its effectiveness for producing compositions and devices composed of one or more polymeric matrix materials and one or more biologically active agents. Solid-state shear pulverization (hereafter, “SSSP”) and solid-state melt-extrusion (hereafter, “SSME”) techniques, however, may achieve better dispersion of heterogeneous nucleating agents in homo-polymers compared to TSE processes. Such techniques may, therefore, be useful for forming well-dispersed compositions of biologically active agents in biocompatible polymeric matrix materials.

A polymeric matrix material may be composed of a polymeric mixture. In some embodiments, the polymeric mixture may be composed of one or more of a homo-polymer, a polymer blend, a combination of a polymer and a filler, and a combination of a polymer and a nanofiller. In some non-limiting examples, the polymeric mixture may be composed of one or more homo-polymers such as a polyolefin, a polyester, a polyamide, an epoxy, and an elastomer or a co-polymer of a polyolefin, a polyester, or a polyamide.

In other non-limiting examples, the polymeric mixture may be composed of a polymer and a filler, in which the filler may be composed of one or more of a cellulose material, a rice husk ash, a talc material, a silica material, a clay material, a modified clay material, a graphite material, a modified graphite material, a graphene, a single-walled carbon nanotube material, a multi-walled carbon nanotube material, and a contrast material for a biological imaging procedure such as barium sulfate. In other non-limiting examples, the polymeric mixture may be composed of a polymer and a nano-filler in which the nano-filler may be composed of one or more of a cellulose material, a rice husk ash, a talc material, a silica material, a clay material, a modified clay material, a graphite material, a modified graphite material, a graphene, a single-walled carbon nanotube material, and a multi-walled carbon nanotube material. Nano-fillers may be distinguished from fillers in that the nano-fillers may have particle sizes of about 1 nm to about 100 nm while fillers may have particle sizes of about 100 μm to about 1 cm. The amount of filler included in a polymeric mixture may range from about 0.001% by weight to about 99% by weight.

In some non-limiting embodiments, such polymeric matrix materials may include one or more biocompatible polymers. Non-limiting examples of such biocompatible polymeric matrix materials may include one or more of a polyolefin, a polyurethane, and a polyether ether ketone. Non-limiting examples of such biocompatible polyolefins may include high density polyethylene and polypropylene. In other non-limiting examples, the polymeric matrix material may be composed of a biocompatible polymer and a biocompatible filler. Non-limiting examples of such biocompatible fillers may include one or more of a cellulose material, a rice husk ash, a talc material, a silica material, a clay material, a modified clay material, a graphite material, a modified graphite material, a graphene, a single-walled carbon nanotube material, a multi-walled carbon nanotube material, and one or more contrast materials for biological imaging procedures, such as barium sulfate. In other non-limiting examples, the polymeric matrix material may be composed of a biocompatible polymer and a biocompatible nano-filler. Non-limiting examples of such biocompatible nano-fillers may include one or more of a cellulose material, a rice husk ash, a talc material, a silica material, a clay material, a modified clay material, a graphite material, a modified graphite material, a graphene, a single-walled carbon nanotube material, a multi-walled carbon nanotube material, and contrast materials for biological imaging procedures. Additional nano-fillers may include metal/metal-oxide nanoparticles having an average size of about 100 nm or less. Non-limiting examples of such metal/metal-oxide nanoparticles may include gold nanoparticles, silver nanoparticles, and titanium dioxide nanoparticles.

In some non-limiting embodiments, a biologically active agent may include one or more of a small organic molecule, a macro molecule, a biological co-factor, a peptide, a protein, and a nucleic acid. Non-limiting examples of such biologically active agents may include one or more of an anti-inflammatory agent, an angiogenic molecule, an anti-infective agent, an anesthetic, a growth factor, an adjuvant, a wound healing factor, a resorbable device component, an immunosuppressive agent, an antiplatelet agent, an anticoagulant, an ACE inhibitor, a cytotoxic agent, an anti-barrier cell compound, a vascularization compound, and an anti-sense nucleic acid. Non-limiting examples of anti-inflammatory agents that may be used may include one or more of steroidal agents (such as dexamethasone and prednisolone) and non-steroidal agents (such as acetyl salicylic acid, acetaminophen, ibuprofen, naproxen, and piroxicam). Non-limiting examples of angiogenic molecules may include one or more of sphingosine-1-phosphate and monobutyrin. Non-limiting examples of immunosuppressive agents may include one or more of cyclosporin A, and rapamycin and its derivatives such as CCI-779, RAD001, and AP23576. In some embodiments, the bioactive agent may include one or more of monobutyrin, S1P (sphingosine-1-phosphate), cyclosporin A, anti-thrombospondin-2, rapamycin (and its derivatives), and dexamethasone. In some non-limiting embodiments, the biologically active agent may be one or more of an anti-inflammatory agent and an angiogenic molecule. In some alternative embodiments, the bioactive agent may include one or more small bioactive molecules such as, but not limited to, monobutyrin.

Disclosed herein are compositions and devices for the delivery of one or more biologically active agents to a subject. Such delivery devices of biologically active agents may include implantable medical devices and wound dressings. Such implantable medical devices and wound dressings may include one or more biologically active agent delivery compositions. Further disclosed herein are biologically active agent delivery devices which may reduce or suppress adverse biological responses associated with implantable devices. In one aspect, the delivery devices may promote vascularization in tissues surrounding the implanted device. In another aspect, these compositions or devices can be designed to vary the rate of delivery of bioactive molecules with a change in the physiological environment surrounding the device. In one embodiment, a biologically active agent delivery device may be composed only of the biologically active agent delivery composition. In an alternative embodiment, such delivery devices may be composed of the composition as well as the matrix material alone. In yet another embodiment, such delivery devices may further include additional components or materials along with the composition and the matrix material. The devices and compositions disclosed herein can be used to deliver a wide variety of biologically active agents.

Many typical methods for fabricating such compositions may rely on heating the matrix material to a liquid or semi-liquid state, mixing biologically active agents with the melt, and then cooling the mixture to a solid state. Alternatively, the matrix material and biologically active agents may be co-dissolved in a solvent which then may be removed during processing. Such methods may not result in well-dispersed bioactive agents in the matrix. Solvent methods may require the use of solvents having high temperatures of vaporization to dissolve the matrix material. Solvent removal through evaporative means may thus require exposing the agents to temperatures that may degrade or destabilize the agents. Additionally, some matrix materials may only be dissolved at temperatures near their liquefication temperatures, thus exposing the biologically active agent to excessive temperatures. However, SSSP and SSME techniques may provide a method for solid admixture of one or more biologically active agents with one or more polymeric matrix materials to form the biologically active composition that may overcome at least some of these limitations.

SSSP techniques alone may result in the temperature of the one or more matrix materials rising above their liquefication temperatures. Such heating may result from the mechanical action of the pulverizing and mixing elements on the matrix material thereby leading to frictional heating to temperatures above the liquefication temperatures of the matrix materials. If the biocompatible polymers become heated above their liquefication temperatures, they may form a melt in which the one or more biologically active agents may be poorly dispersed. Thus, temperature control of the solid state pulveriving systems may be used to maintain all of the components of the system at or below the liquefication temperature of the polymer matrix materials.

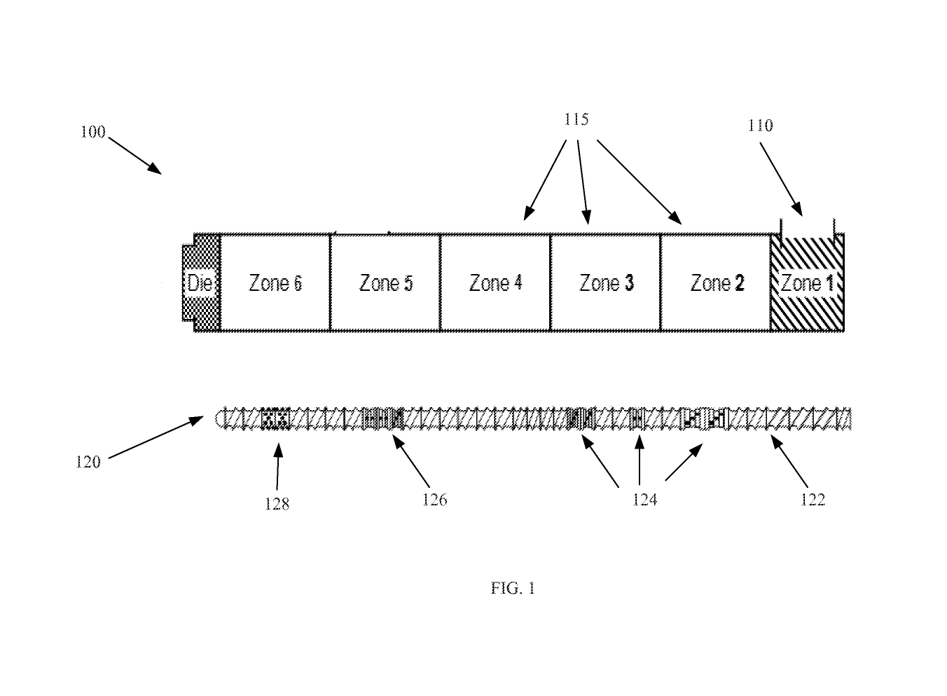

FIG. 1 depicts a non-limiting configuration of a system for fabricating a biologically active agent delivery composition. Such a system may include a screw extruder including one or more extrusion screws. In FIG. 1, an extrusion screw 120 may be housed within an enclosure 100 that maintains physical contact between the materials being processed and the active elements of the extrusion screw. The extrusion screw 120 may be composed of a shaft and modular elements or may be a monolithic structure. The extrusion screw 120 may be composed of any material having physical characteristics capable of manipulating the polymeric materials and the biologically active agents, including, without limitation, stainless steel, aluminum, iron, high carbon steel, tempered steel, and surface-hardened metals.

Non-limiting examples of the active elements of the extrusion screw 120 may include one or more transport elements 122, mixing elements 124, and pulverizing elements 126, 128. The order, number, or type of the active elements along the extrusion screw 120 may not be limited to the configuration as depicted in FIG. 1, but may include any order of elements as may be required to transport, mix, combine, pulverize, or otherwise manipulate the materials introduced into the system for fabricating a biologically active agent delivery composition. For example, additional active elements may be included to knead the composition. Starting materials may be introduced into the extruder at one or more feed chutes 110 of the enclosure 100. Starting materials may include one or more of a polymeric matrix material, a filler, a nanofiller, and a biologically active agent. The starting materials may be introduced as a combination of one or more of the polymeric matrix material, the filler, the nanofiller, and the biologically active agent through a single feed chute 110. The starting materials may be added as a single combined material or as individual components of matrix, filler, and agent added sequentially in any order. Alternatively, each individual component of matrix, filler, and agent may be added via its own feed chute 110 or via any combination of one or more feed chutes.

The starting materials, once introduced into the solid-state shear pulverization system, may travel continuously along the length of the enclosure 100 due to the continuous rotation of the extrusion screw 120 and its effects on the active elements 122, 124, 126, 128. The final biologically active agent delivery composition may be delivered by the extrusion screw 120 to a die end configured to dispense the final particulate composition. In this manner, the biologically active agent delivery composition may be continuously processed from introduction of the starting materials into the screw extruder to the receipt of the final particulate composition. Along the length of the extrusion screw 120, the initial mixture of materials may be subjected to mixing, grinding, and pulverizing forces generated by the mixing elements 124, pulverizing elements 126, 128, or other elements as required to achieve the required blending of materials and sizing of the final particulate material.

Although FIG. 1 illustrates a single extrusion screw 120, a system for fabricating a biologically active agent composition may be composed of one or more extrusion screws. In some embodiments, the system may have a plurality of extrusion screws 120 configured so that their active elements may interact to improve grinding or mixing the material. An example of such a device may be a twin extrusion screw extruder having a pair of extrusion screws proximate to each other and having their respective screw axes effectively parallel to each other.

The enclosure 100 may be divided into effective work zones, as depicted in FIG. 1 (see Zone 1-Zone 6). Such work zones may be defined in terms of the processing steps of the composition and/or the temperature of the composition within them. Thus, Zone 1 may correspond to a section in which the starting materials are introduced into the extruder via one or more feed chutes 110. One or more initial zones (for example Zone 2 and Zone 3) may correspond to sections in which the starting materials are subjected to the action of the mixing elements 124. A buffer zone Zone 4 may be set between a mixing process and a pulverizing process occurring in one or more pulverizing zones (for example Zone 5 and Zone 6) in which the pulverizing elements 126, 128 may operate, respectively. In one embodiment, it may be understood that the one or more initial zones may incorporate all those work zones Zone 2-Zone 6 in which the starting materials and/or composition may be mixed, pulverized, kneaded, or otherwise physically manipulated.

It may be understood that the starting materials may be introduced into the SSSP via one or more feed chutes 110 that may deliver all the starting materials into one work zone. Alternatively, some of the starting materials, such as the polymer matrix material, may be introduced in one work zone such as Zone 1, while other starting materials, such as the biologically active agent, may be introduced in a different work zone such as Zone 2.

Work zones Zone 1-Zone 6 may also be defined functionally in terms of their operating temperatures or the mechanical processes occurring therein. Non-limiting examples of such work zones may have physical embodiments as barrel sections (for example, 115). Barrel sections 115 may be composed of segments of metal or other materials that physically surround one or more sections of the extruder screw 120 and one or more active elements such as mixing elements 124. In one non-limiting example, the enclosure 100 may be composed of one or more barrel sections 115 linked together. In another non-limiting example, the one or more barrel sections 115 may be separate structural elements contained within the enclosure 100. The one or more barrel sections 115 may be composed of any suitable material including, without limitation, stainless steel, aluminum, iron, high carbon steel, tempered steel, and surface-hardened metals.

It may be understood that the configuration of the extruder screw 120 and the active elements as disclosed in FIG. 1 is illustrative only and is not intended to limit the possible configurations of the extruder screw or of its components. Similarly, the descriptions of the work zones or barrel sections 115 in FIG. 1 are illustrative only and are not intended to suggest a single set of temperatures, activities, number, or relative locations of such work zones.

As disclosed above, frictional heating of the composition during processing may lead to the mixture being heated to or above a liquefication temperature of at least some component of the mixture, such as a polymeric matrix material. Such frictional heating and liquefication may result in inhomogeneous mixing of the polymeric matrix material and the biologically active agent. Thus, in one embodiment, the temperature of the at least one extrusion screw 120 of the extruder may be controlled to remove at least some of the friction-induced heat from the composition. In one non-limiting embodiment, the temperature of the at least one extrusion screw 120 may be maintained at a temperature less than or equal to the liquefication temperature of the polymeric matrix material. Table I presents exemplary polymeric matrix materials and their liquefication temperatures.

TABLE I

| Polymeric | | Liquefication | Liquefication |
|---|---|---|---|
| Matrix | Type of | Temperature | Temperature |
| Material | Material | (Melting Point) | (Glass Transition) |
| Polyethylene | Amorphous/Semi- | 248° F.-356° F. | −130° F. |
| (high density) | crystalline | (120° C.-180° C.) | (−90° C.) |
| Poly Ether | Amorphous/Semi- | 662° F.-716° F. | 302° F. |
| Ether Ketone | crystalline | (350° C.-380° C.) | (150° C.) |
| Polyurethane | Amorphous/Semi- | 302° F.-401° F. | −18° F. |
| | crystalline | (150° C.-205° C.) | (−28° C.) |
| Polypropylene | Amorphous/Semi- | 266° F.-340° F. | 6.8° F. |
| | crystalline | (130° C.-171° C.) | (−14° C.) |
| Polyethylene | Amorphous/Semi- | 482° F.-500° F. | 158° F. |
| terephthalate | crystalline | (250-260° C.) | (70° C.) |
| Nylon 6,6 | Amorphous/Semi- | 491° F.-518° F. | 122° F. |
| | crystalline | (255-270° C.) | (50° C.) |
| Polycarbonate | Amorphous | N/A | 297° F. |
| | | | (147° C.) |

In some non-limiting examples, the temperature of at least one portion of the at least one extrusion screw 120 may be maintained at a temperature of about 35° F. to about 45° F. (about 1.7° C. to about 7.2° C.). Some non-limiting examples of temperatures at which the at least one portion of the at least one extrusion screw 120 may be maintained may include a temperature of about 35° F. (about 1.7° C.), about 37° F. (about 2.8° C.), about 39° F. (about 3.9° C.), about 40° F. (about 4.4° C.), about 42° F. (about 5.6° C.), about 44° F. (about 6.7° C.), about 45° F. (about 7.2° C.), or ranges between any two of these values including endpoints. As one example, the one or more extrusion screw 120 may be maintained at a temperature of about 40° F. (about 4.4° C.). Because the polymeric matrix materials may not have high thermal conductivity, the extrusion screw 120 may be maintained at temperatures significantly lower than the liquefication temperature of the biocompatible matrix material in order to maintain the matrix material in a solid state. For example, it may be necessary to maintain the extrusion screw 120 temperature at about 12° F. (about −11° C.) in order to maintain the polymeric materials at about 38° F. (about 3.3° C.) during the manipulation steps of the extruder.

It may be understood that the material in any of the one or more work zones or barrel sections 115 in an SSSP device as illustrated in FIG. 1 may be maintained at a temperature equal to or less than a liquefication temperature of any of the components, for example of the biocompatible matrix mixture. Such work zones or barrel sections 115 may include, without limitation, a work zone in which the starting materials are introduced into the extruder (for example, Zone 1), one or more initial zones (for example, Zone 2 and Zone 3), a buffer zone (for example Zone 4), one or more pulverizing zones (for example, Zone 5 and Zone 6), and a delivery zone (for example, Die).

The granular form of the biologically active agent delivery composition produced under the conditions disclosed above may have particle sizes less than about 1 μm. In some non-limiting examples, the particulates composed of the polymer matrix material may be about 1 μm or less. In some non-limiting examples, the particulates composed of the biologically active agent may be about 1 μm or less. In other non-limiting examples, the particulates composed of the biologically active agent may be about 100 nm or less. In still other non-limiting examples, the particulates composed of the biologically active agent may be about 10 nanometers or less. In general, the biologically active agent delivery composition may be composed of particulates of the biologically active agent evenly dispersed throughout the composition and not aggregated in clumps.

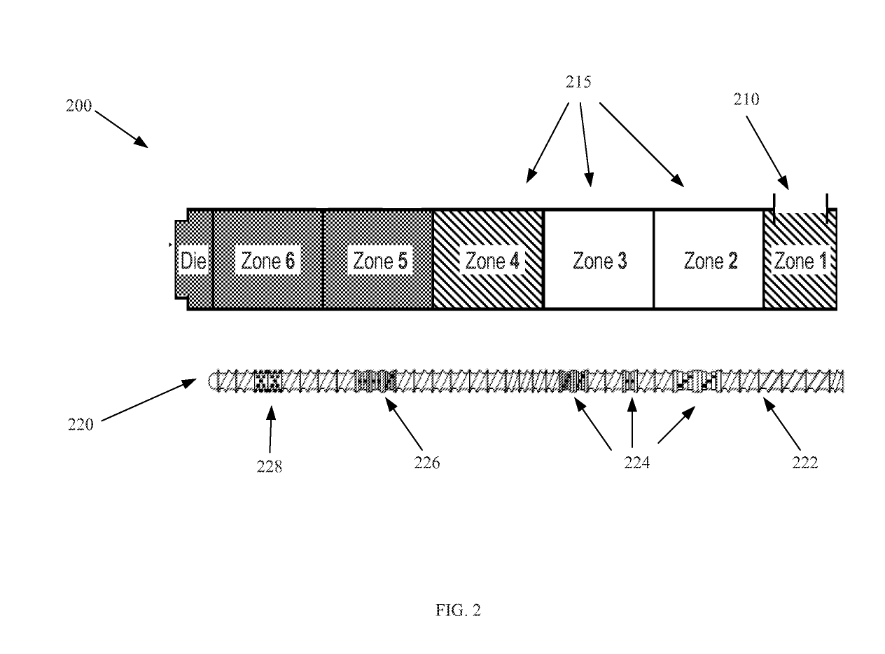

FIG. 2 depicts a non-limiting configuration of an SSME device. In FIG. 2, an extrusion screw 220 is housed within an enclosure 200 that maintains physical contact between the mixture being processed and the active elements of the extrusion screw. The extrusion screw 220 may be composed of a shaft and modular elements, or may be a monolithic structure. The extrusion screw 220 may be composed of any material having physical characteristics capable of manipulating the starting materials, including, without limitation, stainless steel, aluminum, iron, high carbon steel, tempered steel, and surface-hardened metals.

Non-limiting examples of the active elements of the extrusion screw 220 may include one or more transport elements 222, pulverizing elements 224, kneading elements 226, and mixing elements 228. The order, number, or type of the active elements along the extrusion screw 220 may not be limited to the configuration as depicted in FIG. 2, but may include any order of elements as may be required to transport, mix, combine, pulverize, or otherwise manipulate the starting materials introduced into the SSME. Such starting materials may include, without limitation, one or more biocompatible polymers, one or more fillers, and one or more biologically active agents. It may be further understood that continuous operation (such as rotation) of the extrusion screw 220 may result in the starting material, introduced at one or more feed chutes 210 of the enclosure 200, to travel continuously along the length of the enclosure to a die end configured to extrude the final fluid mixture. In this manner, the mixture of the one or more biocompatible polymers and one or more biologically active agents may be continuously processed from introduction of the starting materials into the screw extruder to the receipt of the extruded fluid material composition. Along the length of the extrusion screw 220, the starting materials may be subjected to mixing, grinding, and pulverizing forces generated by the pulverizing elements 224, kneading elements 226, mixing elements 228, or other elements as required to achieve the required blending of materials. It may be understood that the starting materials may be introduces together as a mixture or separately. The starting materials may also be introduced at a single feed chute 210 as a mixture or by sequential addition. Additionally, the starting materials may be introduced separately at one or more individual feed chutes 210. The individual feed chutes 210 may deliver their respective contents to the same work zone or to different work zones along the enclosure 200.

Although FIG. 2 illustrates a single extrusion screw 220, an SSME device may be composed of one or more extrusion screws. In some embodiments, an SSME device may have a plurality of extrusion screws 220 configured so that their active elements may interact to improve grinding or mixing the matrix material with the one or more biologically active agents. An example of such a device may be a twin extrusion screw extruder having a pair of extrusion screws proximate to each other and having their respective screw axes effectively parallel to each other.

The enclosure 200 in which the one or more extrusion screws 220 are housed may be divided into effective work zones, as depicted in FIG. 2 (see Zone 1-Zone 6). Such work zones may be defined in terms of their respective temperatures and/or the processing steps of the material within them. Thus, Zone 1 may correspond to a section in which the starting materials are introduced into the extruder via one or more feed chutes 210 at an ambient temperature. One or more initial zones (for example, Zone 2 and Zone 3) may correspond to sections in which the starting material may be subjected to the action of the pulverizing elements 224 thereby producing a sheared mixture of the starting material. During the pulverization process, the material may be kept at a temperature at or below the liquefication temperature of the biocompatible polymer material. Thus, the one or more initial zones (Zone 2 and Zone 3) may include temperature control elements (for example, as part of the one or more extrusion screws 220) to maintain the temperature of the starting material in those work zones at or below the liquefication temperature of the biocompatible polymeric material. Transition zone Zone 4 may be a buffer zone between the pulverizing process in the one or more initial zones (Zone 2 and Zone 3) and the kneading process occurring in one or more heating zones (for example Zone 5).

While the SSSP process produces particulate material, the SSME process incorporates an additional melt extrusion step. Consequently, the SSME extruder depicted in FIG. 2 includes additional processing steps to melt the particulate biologically active agent delivery composition. The melted composition may be extruded to form any one or more products, including, without limitation, a wire, a sheet, a tube, a multi-lumen tube, or any other profile extruded from a die as commonly known to those having ordinary skill in the art. In some embodiments, the extruded melted composition may be further processed to form biologically active agent delivery devices. In some alternative embodiments, the extruded melted composition may itself be used as the biologically active agent delivery device. The melting process may occur for example in one or more heating zones (for example, in Zone 5 and Zone 6) in which the kneading elements 226 and mixing elements 228 may operate, respectively. The temperature in the zones manipulating the melted biologically active agent delivery composition may be greater than or equal to a liquefication temperature of the polymeric mixture. Because the biologically active agent delivery composition produced in the one or more initial zones (Zone 2 and Zone 3) may be at a temperature at or below the liquefication temperature of the polymeric mixture, and the melted composition in the one or more heating zones (Zone 5 and Zone 6) may be at a temperature at or above the liquefication temperature of the polymeric mixture, the biologically active agent delivery composition transported from Zone 3 to Zone 5 may be at an intermediate temperature as it is transported through the transition zone Zone 4. As a non-limiting example, the starting materials in the one or more initial zones (Zone 2 and Zone 3) may be maintained at a temperature of about 38° F. (about 3.3° C.), the melted agent delivery composition in the one or more heating zones (Zone 5 and Zone 6) may be maintained at a temperature of about 400° F. (about 204° C.), and the transported delivery composition may have an average temperature of about 70° F. (about 21° C.) as it transits through transition zone Zone 4. It may be appreciated that the composition may be warmed from a temperature at or below a liquefication temperature to a temperature at or above the liquefication temperature of the polymer as it is transferred through the transition zone.

Work zones Zone 1-Zone 6 may be defined functionally in terms of their operating temperatures or the mechanical processes occurring therein. Non-limiting examples of such work zones may have physical embodiments as barrel sections (for example, 215). Barrel sections 215 may be composed of segments of metal or other materials that may physically surround one or more sections of the extruder screw 220 and one or more active elements such as pulverizing elements 224. In one non-limiting example, the enclosure 200 may be composed of one or more barrel sections 215 linked together. In another non-limiting example, the one or more barrel sections 215 may be separate structural elements contained within the enclosure 200. The one or more barrel sections 215 may be composed of any suitable material including, without limitation, stainless steel, aluminum, iron, high carbon steel, tempered steel, and surface-hardened metals.

It may be understood that the configuration of the extruder screw 220 and the active elements as disclosed in FIG. 2 is illustrative only and is not intended to limit the possible configurations of the extruder screw or of its components. Similarly, the descriptions of the work zones and barrel sections 215 in FIG. 2 are illustrative only and are not intended to suggest a single set of temperatures, activities, number, or relative locations of such work zones.

As disclosed above, frictional heating of the combination of one or more biocompatible polymer materials and one or more biologically active agents during processing may lead to the mixture being heated to or above a liquefication temperature of at least some component of the combination. Such frictional heating and liquefication may result in inhomogeneous mixing of the one or more biologically active agents into the biocompatible polymer material during pulverization. In one non-limiting embodiment, the temperature of one or more portions of the at least one extrusion screw 220 having active elements that may pulverize the starting material (for example, in one or more initial zones such as Zone 2 and Zone 3) may be maintained at a temperature less than or equal to the liquefication temperature of the biocompatible polymer material. In some non-limiting examples, the temperature of the one or more portions of the at least one extrusion screw 220 having active elements to pulverize the starting material may be maintained at a temperature of about 35° F. to about 45° F. (about 1.7° C. to about 7.2° C.). Some non-limiting examples of temperatures at which at least one portion of the at least one extrusion screw 220 may be maintained may include a temperature of about 35° F. (about 1.7° C.), about 37° F. (about 2.8° C.), about 39° F. (about 3.9° C.), about 40° F. (about 4.4° C.), about 42° F. (about 5.6° C.), about 44° F. (about 6.7° C.), about 45° F. (about 7.2° C.), or ranges between any two of these values including endpoints.

Similarly, the temperature of one or more portions of the at least one extrusion screw 220 having active elements to mix or knead the melted biologically active agent delivery composition (for example, in one or more heating zones such as Zone 5 and Zone 6) may be maintained at a temperature greater than or equal to the liquefication temperature of the biocompatible polymer material. In some non-limiting examples, the temperature of one or more portions of the at least one extrusion screw 220 having active elements to mix or knead the melted particulate biologically active agent delivery composition may be maintained at a temperature of about 90° F. to about 500° F. (about 32° C. to about 260° C.). Some non-limiting examples of temperatures at which the at least one extrusion screw 220 may be maintained to mix or knead the melted polymer mixture may include a temperature of about 90° F. (about 32° C.), about 199° F. (about 93° C.), about 250° F. (about 121° C.), about 300° F. (about 149° C.), about 351° F. (about 177° C.), about 399° F. (about 204° C.), about 450° F. (about 232° C.), about 500° F. (about 260° C.), or ranges between any two of these values including endpoints. In other non-limiting examples, the melted composition may be maintained at a temperature just sufficient to cause phase melting of the polymer matrix.

It may be understood that temperature control, such as cooling, of the polymeric matrix materials, bioactive agents, and filler materials, either separately or in any combination throughout the manipulations by the screw extrusion device may be accomplished by any appropriate means.

Cooling may be accomplished by cooling one or more portions of the extrusion screw according to the type of manipulation of the material contacting the extrusion screw (for example, in one or more initial zones such as Zone 2 and Zone 3 in FIG. 2). A portion of the enclosure 100 (FIG. 1) or 200 (FIG. 2) encompassing the extrusion screw or barrel sections 115 (FIG. 1) or 215 (FIG. 2) may also be cooled according to the type of manipulation of the material therein (for example, in Zone 2, Zone 3, Zone 4, and Zone 5 in FIG. 1). Such cooling may be accomplished through the use of one or more of a heat exchange coil, a compressor, a refrigerator, and a solid state cooling device through a temperature control system. In one non-limiting example, heat exchange tubing may be placed in thermal contact with one or more of portions of the one or more extrusion screws 120, 220, one or more active elements 124, 126, 128, 224, 226, and 228, one or more barrel sections 115, 215, and one or more sections of the enclosure 100, 200. The heat exchange tubing may be filled with a recirculating refrigeration liquid such as a mixture of water and ethylene glycol. The refrigeration liquid may be kept at a constant temperature according to devices and control systems as are known in the art.

It may be understood that the one or more portions of the enclosure 100, 200, extrusion screw 120, 220, barrel sections 115, 250, and active elements 124, 126, 128, 224, 226, and 228, may be controlled to have any appropriate temperature such as a temperature at or below a liquefication temperature of one or more components of the polymer matrix materials. It may further be understood that each of the one or more portions of the enclosure 100, 200, extrusion screw 120, 220, barrel sections 115, 215, and active elements 124, 126, 128, 224, 226, and 228, may be controlled to have about the same temperature or a different temperature. In some non-limiting examples, the one or more portions of the enclosure 100, 200, extrusion screw 120, 220, barrel sections 115, 215, and active elements 124, 126, 128, 224, 226, and 228, may be controlled to have a temperature less than or equal to about 40° C. In some other non-limiting examples, the one or more portions of the enclosure 100, 200, extrusion screw 120, 220, barrel sections 115, 215, and active elements 124, 126, 128, 224, 226, and 228, may be controlled to have a temperature of about 35° C. to about 45° C.

With respect to SSME processing, heating of the particulate form of the biologically active agent delivery composition may be accomplished by heating one or more portions of the extrusion screw according to the type of manipulation of the polymeric material contacting the extrusion screw (for example, in one or more heating zones such as Zone 5 and Zone 6 in FIG. 2). A portion of the physical enclosure 200 of the extrusion screw or barrel section 215 may also be heated according to the type of manipulation of the polymeric material therein (for example, one or more heating zones such as Zone 5 and Zone 6 in FIG. 2). Such heating may be accomplished through the use of one or more of a resistive heating element, a heat transfer coil, a radiant heating device, and the introduction of a heated gas. Because some portions of an SSME processing device may be kept at a temperature at or above a liquefication temperature of the biocompatible polymer material, while other portions may be kept at a temperature at or below a liquefication temperature, thermal insulating components or devices may be required to provide thermal barriers between the high temperature and low temperature portions of the physical enclosure 200 or between barrel sections 215.

A biologically active agent delivery device may be fabricated from the biologically active agent delivery composition. Fabrication methods may include those best suited for the type of delivery device and the form of the delivery composition. In one non-limiting example, a delivery composition may be fabricated as a fine particulate material having an average particle diameter of about 1 μm to about 10 μm. Some non-limiting examples an average particle diameter may include a diameter of about 1 μm, about 2 μm, about 3 μm, about 4 μm, about 5 μm, about 6 μm, about 7 μm, about 8 μm, about 9 μm, about 10 μm, or ranges between any two of these values including endpoints.

Such particulate forms of the delivery composition may be fabricated into a delivery device using any useful fabrication techniques including, without limitation, one or more of melt extrusion techniques, injection molding techniques, and compression molding techniques. Delivery devices fabricated from the particulate form of delivery compositions may have any useful form including, without limitation, a ring, a pill, a tube, a multilumen tube, a straight cylinder, and a curved cylinder. Alternatively, particulate forms of the delivery composition may be included directly in wound dressings such as sponges, bandages, gauzes, and similar structures used on superficial wounds. In another non-limiting example, the particulate form of the biologically active agent delivery composition may be compounded with a liquid carrier for injection into a body such as, for example, for intraperitoneal injection. Such particulate forms of active agent delivery compositions may further be incorporated into a variety of implanted biomedical devices including, without limitation, stents, internal sutures, intrauterine devices, and electrostimulation leads.

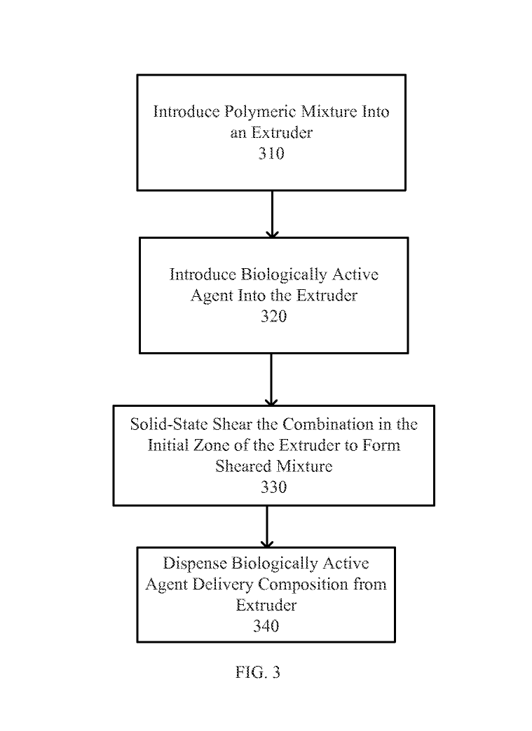

FIG. 3 is a flow chart of an exemplary method for producing a particulate biologically active agent delivery composition using an SSSP device. One or more biocompatible polymer matrix materials may be introduced 310 into an extruder, such as a twin-screw extruder. One or more biologically active agents may be introduced 320 into the extruder. The one or more biocompatible polymer matrix materials and one or more biologically active agents may be introduced separately or together into the extruder. The one or more biocompatible polymer matrix materials and one or more biologically active agents together may comprise one or more starting materials. A sheared mixture of starting materials may be produced in at least an initial zone of the extruder by means of solid-state shearing 330. Such a sheared mixture may be fabricated by any combination of mixing, pulverizing, or kneading the starting material by one or more active elements of the extruder. The sheared mixture may be dispensed 340 from the extruder at a dispensing end as a particulate biologically active agent delivery composition.

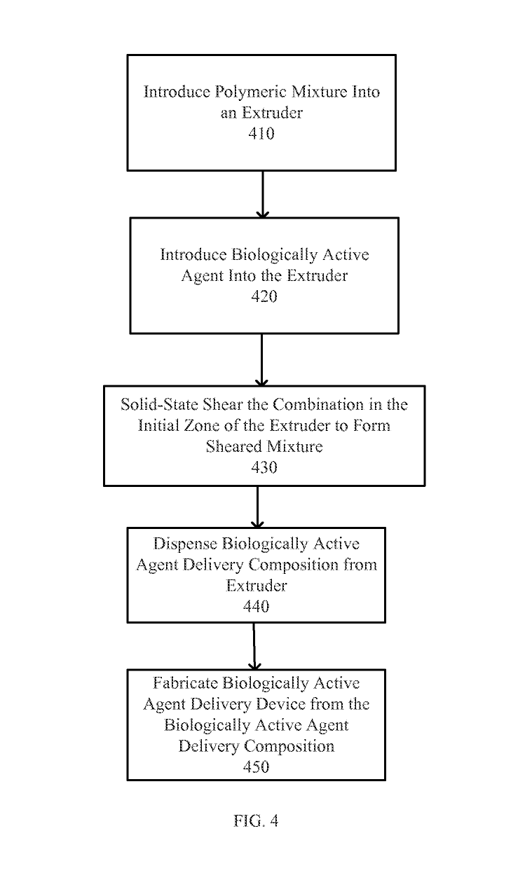

FIG. 4 is a flow chart of an exemplary method for producing a biologically active agent delivery device using an SSSP device. One or more biocompatible polymer matrix materials may be introduced 410 into an extruder, such as a twin-screw extruder. One or more biologically active agents may be introduced 420 into the extruder. The one or more biocompatible polymer matrix materials and one or more biologically active agents may be introduced separately or together into the extruder. The one or more biocompatible polymer matrix materials and one or more biologically active agents together may comprise one or more starting materials. A sheared mixture starting material may be produced in at least an initial zone of the extruder by means of solid-state shearing 430. Such a sheared mixture may be fabricated by any combination of mixing, pulverizing, or kneading the starting material by one or more active elements of the extruder. The sheared mixture may be dispensed 440 from the extruder at a dispensing end as a particulate biologically active agent delivery composition. The particulate biologically active agent delivery composition may be fabricated 450 into any appropriate type of biologically active agent delivery device.

### EXAMPLES

### Example 1

### Exemplary Compositions of Polymeric Matrix Materials and Biologically Active Agents

Table II presents non-limiting examples of compositions of polymeric matrix materials and biologically active agents that may be used according to the methods disclosed herein (values presented as weight percent of a total combination).

TABLE II

| | Material | | | | | | |
|---|---|---|---|---|---|---|---|
| | Poly- | Poly ether | Poly- | Mono- | Acet- | Rapa- | Clay |
| | ethylene | ether ketone | urethane² | butyrin | aminophen | mycin | filler |
| | (wt %) | (wt %) | (wt %) | (wt %) | (wt %) | (wt %) | (wt %) |
| Sample 1 | 89 | 0 | 0 | 1 | 0 | 0 | 10 |
| Sample 2 | 89 | 0 | 0 | 0 | 1 | 0 | 10 |
| Sample 3 | 89 | 0 | 0 | 0 | 0 | 1 | 10 |
| Sample 4 | 0 | 89 | 0 | 1 | 0 | 0 | 10 |
| Sample 5 | 0 | 89 | 0 | 0 | 1 | 0 | 10 |
| Sample 6 | 0 | 89 | 0 | 0 | 0 | 1 | 10 |
| Sample 7 | 0 | 0 | 74 | 1 | 0 | 0 | 25 |
| Sample 8 | 0 | 0 | 74 | 0 | 1 | 0 | 25 |
| Sample 9 | 0 | 0 | 74 | 0 | 0 | 1 | 25 |
| Sample 10 | 99.97 | 0 | 0 | 0.01 | 0.01 | 0.01 | 0 |
| Sample 11 | 0 | 99.97 | 0 | 0.01 | 0.01 | 0.01 | 0 |
| Sample 12 | 0 | 0 | 99.97 | 0.01 | 0.01 | 0.01 | 0 |
| Sample 13 | 0 | 0 | 65 | 3 | 2 | 0 | 30 |
| Sample 14 | 0 | 0 | 65 | 0 | 3 | 2 | 30 |
| Sample 15 | 0 | 0 | 60 | 0 | 25 | 0 | 15 |

### Example 2

### Exemplary Methods of and Systems for Fabricating Compositions of Polymeric Matrix Materials and Biologically Active Agents

Solid-state shear pulverization (SSSP) was performed using an intermeshing, co-rotating twin screw extruder with a diameter (D) of about 25 mm and a length to diameter ratio (L/D) of about 34. The screws were modular in nature and designed as a combination of spiral conveying and bilobe kneading/pulverization elements. For the SSSP apparatus, all of the barrels were continuously cooled by recirculating ethylene glycol/water (60/40 vol/vol) mixture maintained at about −12° C. Polymers, fillers, and/or biologically active agents were delivered to the extruder using constant volume feeders. The barrel sections of the extruder included several kneading elements in an upstream portion of the screws termed the mixing zone. The material exited the mixing zone through a conveying zone that allowed the sheared material to cool before being pulverized downstream in a pulverization zone.

For the SSME apparatus, the barrel temperature was customized to create three distinct zones along the length of the barrel. Zone 1, spanning the beginning length having a L/D ratio of about 16, was designed for solid-state pulverization. This portion of the barrel was continuously cooled at about −12° C. by a circulating ethylene glycol/water mixture. Zone 2 (having an L/D ratio of about 6) included an intermediate barrel maintained at a temperature of about 21° C., where the materials transitioned from the solid-state to a melted-state. Finally, Zone 3 (having an L/D ratio of about 12) included the melt extrusion zone wherein the barrel was heated to about 204° C. by standard cartridge-type electrical heaters. The system incorporated spiral transporting elements (having an L/D ratio of about 8.5) and bilobe kneading elements (having an L/D ratio of about 7.5) in Zone 1, all spiral transporting elements in Zone 2, and spiral transporting elements (having an L/D ratio of about 8.3) and bilobe shearing and mixing elements (having an L/D ratio of about 3.7) in Zone 3. The screw rotation speed was maintained constant at about 200 rpm for set ups.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated in this disclosure, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can, of course, vary. It is also to be understood that the terminology used in this disclosure is for the purpose of describing particular embodiments only, and is not intended to be limiting.

With respect to the use of substantially any plural and/or singular terms in this disclosure, those having skill in the art can translate from the plural to the singular and/or from the singular to the plural as is appropriate to the context and/or application. The various singular/plural permutations may be expressly set forth in this disclosure for sake of clarity.

It will be understood by those within the art that, in general, terms used in this disclosure, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed in this disclosure also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed in this disclosure can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. From the foregoing, it will be appreciated that various embodiments of the present disclosure have been described for purposes of illustration, and that various modifications may be made without departing from the scope and spirit of the present disclosure. Accordingly, the various embodiments disclosed are not intended to be limiting, with the true scope and spirit being indicated by the following claims.

What is claimed is:

1. A method of fabricating a biologically active agent delivery composition, the method comprising:
introducing a polymeric mixture into an extruder;
introducing a biologically active agent into the extruder;
solid-state shearing the polymeric mixture and the biologically active agent together in an initial zone of the extruder to yield the biologically active agent delivery composition, wherein the initial zone has a temperature less than or equal to a liquefication temperature of the polymeric mixture; and
dispensing the biologically active agent delivery composition in a particulate form from the extruder.

2. The method of claim 1, wherein introducing a polymeric mixture into an extruder and introducing a biologically active agent into the extruder comprise introducing a first mixture into the extruder, wherein the first mixture comprises the polymeric mixture and the biologically active agent.

3. The method of claim 1, wherein the extruder comprises a screw extruder having at least one extrusion screw.

4. The method of claim 3, wherein the screw extruder is a twin extrusion screw extruder.

5. The method of claim 3, further comprising controlling a temperature of one or more of at least one section of the at least one extrusion screw, at least one section of an enclosure of the extruder, and at least one barrel section of the extruder.

6. The method of claim 3, further comprising maintaining a temperature of one or more of at least one section of the at least one extrusion screw, at least one section of an enclosure of the extruder, and at least one barrel section of the extruder less than or equal to the liquefication temperature of the polymeric mixture.

7. The method of claim 3, further comprising maintaining a temperature of one or more of at least one section of the at least one extrusion screw, at least one section of an enclosure of the extruder, and at least one barrel section of the extruder less than or equal to about 40° C.

8. The method of claim 3, further comprising maintaining a temperature of one or more of at least one section of the at least one extrusion screw, at least one section of an enclosure of the extruder, and at least one barrel section of the extruder at about 35° C. to about 45° C.

9. The method of claim 3, further comprising controlling a temperature of one or more of at least one barrel section of the screw extruder by contacting the one or more of at least one barrel section with a temperature controlled fluid.

10. The method of claim 3, wherein the screw extruder is a continuously operating screw extruder.

11. The method of claim 1, wherein the polymeric mixture comprises one or more biocompatible polymers, a combination of a biocompatible polymer and a biocompatible filler, and a combination of a biocompatible polymer and a biocompatible nanofiller.

12. The method of claim 1, wherein the polymeric mixture comprises one or more of a polyolefin, a polyether ether ketone, and a polyurethane.

13. The method of claim 1, wherein the polymeric mixture comprises a combination of a biocompatible polymer and a biocompatible filler, wherein the biocompatible filler comprises one or more of a cellulose material, a rice husk ash, a talc material, a silica material, a clay material, a modified clay material, a graphite material, a modified graphite material, a graphene, a single-walled carbon nanotube material, a multi-walled carbon nanotube material, and a contrast material for a biological imaging procedure.

14. The method of claim 1, wherein the polymeric mixture comprises a combination of a polymer and a nano-filler, wherein the nano-filler comprises one or more of a cellulose material, a rice husk ash, a talc material, a silica material, a clay material, a modified clay material, a graphite material, a modified graphite material, a graphene, a single-walled carbon nanotube material, a multi-walled carbon nanotube material, and a contrast material for a biological imaging procedure.

15. The method of claim 1, wherein the biologically active material comprises one or more of a small organic molecule, a macro molecule, a biological co-factor, a peptide, a protein, and a nucleic acid.

16. The method of claim 1, wherein the biologically active material comprises one or more of an anti-inflammatory agent, an angiogenic molecule, an anti-infective agent, an anesthetic, a growth factor, an adjuvant, a wound healing factor, an immunosuppressive agent, an antiplatelet agent, an anticoagulant, an ACE inhibitor, a cytotoxic agent, an anti-barrier cell compound, a vascularization compound, a hormone, and an anti-sense nucleotide.

17. The method of claim 1, wherein the liquefication temperature is a melting point of a semi-crystalline polymer.

18. The method of claim 1, wherein the liquefication temperature is a glass transition temperature of an amorphous polymer.

19. The method of claim 1, wherein solid-state shearing the first mixture further comprises kneading the first mixture.

20. A biologically active agent delivery composition, the composition comprising:
a polymeric mixture; and
a biologically active agent,
wherein the biologically active agent delivery composition is a granular material having an average particle diameter less than or equal to about 100 μm, and
wherein the biologically active agent delivery composition is fabricated by a solid-state shearing device operating at least in part at a temperature less than or equal to a liquefication temperature of the polymeric mixture.

21. The composition of claim 20, wherein the polymeric mixture comprises one or more biocompatible polymers, a combination of a biocompatible polymer and a biocompatible filler, and a combination of a biocompatible polymer and a biocompatible nanofiller.

22. The composition of claim 20, wherein the polymeric mixture comprises one or more of a polyolefin, a polyether ether ketone, and a polyurethane.

23. The composition of claim 20, wherein the biologically active material comprises one or more of a small organic molecule, a macro molecule, a biological co-factor, a peptide, a protein, and a nucleic acid.

24. The composition of claim 20, wherein the biologically active material comprises one or more of an anti-inflammatory agent, an angiogenic molecule, an anti-infective agent, an anesthetic, a growth factor, an adjuvant, a wound healing factor, an immunosuppressive agent, an antiplatelet agent, an anticoagulant, an ACE inhibitor, a cytotoxic agent, an anti-barrier cell compound, a vascularization compound, a hormone, and an anti-sense nucleotide.

25. A biologically active agent delivery device comprising:
a biologically active agent delivery composition, comprising:
a polymeric mixture, and
a biologically active agent,
wherein the biologically active agent delivery composition is a granular material having an average particle diameter less than or equal to about 100 μm, and
wherein the biologically active agent delivery composition is fabricated by a solid-state shearing device operating at least in part at a temperature less than or equal to a liquefication temperature of the polymeric mixture;
wherein the biologically active agent delivery composition is fabricated into the biologically active agent delivery device configured for administration into a portion of a body.

26. The device of claim 25, wherein the portion of the body comprises one or more of a natural bodily cavity, a vascular space, a peritoneal space, a portion of striated muscle, a portion of mucosal tissue, and an optical tissue.

27. The device of claim 25, wherein the biologically active agent delivery device is a ring, a pill, a tube, a multilumen tube, a straight cylinder, or a curved cylinder.

28. A method of fabricating a biologically active agent delivery device, the method comprising:
introducing a polymeric mixture into an extruder;
introducing a biologically active agent into the extruder;
solid-state shearing the polymeric mixture and the biologically active agent together in an initial zone of the extruder to yield the biologically active agent delivery composition, wherein the initial zone has a temperature less than or equal to a liquefication temperature of the polymeric mixture;
dispensing the biologically active agent delivery composition from the extruder; and
fabricating the biologically active agent delivery device from the biologically active agent delivery composition.

29. The method of claim 28, wherein fabricating the biologically active agent delivery device from the biologically active agent delivery composition comprises one or more of extruding the composition, injection molding the composition, and compression molding the composition.

30. A system for fabricating a biologically active agent delivery composition, the system comprising:
at least one barrel section;
at least one extrusion screw disposed within the at least one barrel section;
a plurality of active elements disposed within the at least one barrel section, wherein the active elements are configured to be operated by the at least one extrusion screw;
at least one feed chute configured to deliver one or more of a polymeric mixture and a biologically active agent into the at least one barrel section; and
a temperature control system,
wherein the temperature control system is configured to maintain a temperature of one of more of the one or more barrel sections, the one or more extrusion screws, and the one or more active elements less than or equal to a liquefication temperature of the polymeric mixture.

31. The system of claim 30, wherein the active elements comprise one or more of a transport element, a mixer, a pulverizer, and a kneader.

32. The system of claim 30, wherein the temperature control system comprises a fluid cooling system in thermal contact with one or more of the one or more barrel sections, the one or more extrusion screws, and the one or more active elements.

33. The system of claim 32, wherein the fluid cooling system comprises a cooling system having a fluid mixture of an amount of water and an amount of ethylene glycol.

34. The system of claim 32, wherein the at least one feed chute comprises a plurality of feed chutes.
